(19) United States
(12) Patent Application Publication
(10) Pub. No.: US 2026/0152712 A1
(43) Pub. Date: Jun. 4, 2026

(54) A BIOREACTOR FOR CONTACTLESS CO-CULTURING

(71) Applicant: STELLENBOSCH UNIVERSITY, Stellenbosch, Western Cape Province (ZA)

(72) Inventors: Robert William McClelland POTT, Stellenbosch, Western Cape Province (ZA); Florian Franz BAUER, Stellenbosch, Western Cape Province (ZA); Debra ROSSOUW, Stellenbosch, Western Cape Province (ZA); Rene Kathleen NAIDOO-BLASSOPLES, Stellenbosch, Western Cape Province (ZA); Jennifer Rae OOSTHUIZEN, Stellenbosch, Western Cape Province (ZA)

(21) Appl. No.: 18/686,122

(22) PCT Filed: Aug. 18, 2022
(86) PCT No.: PCT/IB2022/057753

§ 371 Date: Feb. 23, 2024

(30) Foreign Application Priority Data
Aug. 24, 2021 (ZA) 2021/06074

(51) Int. Cl. C12M 1/00; C12M 1/06; C12M 1/34

(52) U.S. Cl. CPC C12M 29/04; C12M 23/58; C12M 27/02; C12M 41/12

(57) ABSTRACT

A co-culture bioreactor is provided and comprises at least two chambers, each configured to house a distinct cell type in a shared culture medium and each having an inlet and an outlet for the shared culture medium. A selectively permeable membrane is associated with each of the at least two chambers and in fluid communication with the outlet of one chamber and an inlet of one or more other chambers. Each of the selectively permeable membranes is individually configured to prevent permeation of cells from the chamber it is associated with into the one or more other chambers whilst allowing culture medium and selected biomolecules to permeate therethrough. A pump is associated with each of the at least two chambers and is configured to circulate culture medium through the selectively permeable membrane and between the at least two chambers.

### CROSS-REFERENCE(S) TO RELATED APPLICATIONS

This application claims priority from South African provisional patent application number 2021/06074 filed on 24 Aug. 2021, which is incorporated by reference herein.

### FIELD OF THE INVENTION

This invention relates to a bioreactor for contactless co-culturing of two or more organism or two or more cell types. In particular it relates to a modular bioreactor for co-culturing two or more organisms or cell types in a shared medium that allows metabolic exchange without physical contact between the cells.

### BACKGROUND TO THE INVENTION

Several biotechnological production processes benefit from co-culture of two or more organisms. Culturing of multispecies systems, either natural or synthetic consortia, provide certain advantages including broader metabolic capabilities, robustness to environmental perturbations, resistance to invasive species and the ability to withstand periods of stress such as nutrient limitation. Certain microbiological processes or studies involve the co-culture of cell types of the same organism that are morphologically or phenotypically distinct or at different life stages. Examination of microbial interactions is a significant and growing research field, requiring the ability to exchange metabolites and other biochemical products or signalling molecules between organisms or cell types without cells being in direct physical contact. Contactless co-culturing processes have many operational difficulties since multispecies or multi-cell type systems, for example, are difficult to control and unpredictable.

Compartmentalised systems can be valuable tools to achieve better control of multispecies/multi-cell type cultures and to study the effects of cell-to-cell contact in co-cultures. Understanding the effects of physical cell-to-cell contact on microbial interactions is essential in the characterisation of microbial communities and their functioning. This is commonly achieved by comparing mixed co-cultures to co-cultures not in direct cell contact while still sharing a common culture medium. Although co-cultures have been shown to provide improved functional and metabolic capabilities in comparison to monocultures, the exact mechanism of the improvement is not always clear. Comparing the effects of co-culturing with and without cell contact between the microbes can assist in understanding whether the interaction is through physical contact or metabolic exchange.

Bioreactors where media is shared between two or more physically separate cell types or microbial populations exist. Current co-culture bioreactors are either based on passive diffusion of biochemical molecules through a single membrane located between two different culture chambers as is the case with the bioreactor system as disclosed in PCT publication WO2007/021919 or involve pumping the culture medium through a filter or membrane between two or more different culture chambers which then requires regular backwashes and or changes in the pumping direction. This can create an uneven flow of metabolites throughout co-culturing and therefore fail to mimic conditions in the natural environment. Some bioreactors with pumps are designed to be monodirectional and transfer culture medium from one culture chamber to the next without allowing metabolic exchange between different cultures. The filters or membranes of such bioreactors are usually configured for supporting tissue growth and harvesting cells on the filter or membrane rather than for purely separating or isolating cell types during a co-culturing process requiring metabolic exchange.

Accordingly, existing membrane technologies rely primarily on transwell systems or on systems where culture is passed through a membrane between two vessels in an alternating manner. Transwell systems rely on passive diffusion of metabolites between two areas with a membrane partition placed between them. Transwell systems are commonly utilized in research as they are very easy to use, and small volumes make them ideal for large-scale screenings. Heyse et al. (Heyse, J., Buysschaert, B., Props, R., Rubbens, P., Skirtach, A. G., Waegeman, W., & Boon, N. (2019). Coculturing bacteria leads to reduced phenotypic heterogeneities. Applied and environmental microbiology, 85(8), e02814-18) used such a system to study the effects of co-culturing bacterial species on phenotypic heterogeneities. The vertical nature of the passive diffusion in such plates prevents the collection of optical density data. Moutinho Jr. et al. (Moutinho Jr, T. J., Panagides, J. C., Biggs, M. B., Medlock, G. L., Kolling, G. L., & Papin, J. A. (2017). Novel co-culture plate enables growth dynamic-based assessment of contact-independent microbial interactions. PloS one, 12(8), e0182163) attempted to solve this using a horizonal membrane placed between two small chambers that once again relies on passive diffusion of culture medium through the membrane layer. Such systems are designed for small volumes of culture, usually no more than 5 ml per vessel. These small volumes are not ideal when running analyses that require large sample volumes and increasing the volume may lead to decrease in the efficiency of diffusion of metabolites in the system.

Manjarrez et al. (Manjarrez, E. S., Albasi, C., & Riba, J. P. (2000). A two-reservoir, hollow-fiber bioreactor for the study of mixed-population dynamics: Design aspects and validation of the approach. Biotechnology and bioengineering, 69(4), 401-408) describe a system still used today whereby pressure on alternating vessel headspaces forces mixing through a hollow fibre membrane module placed between them. This system utilises larger reaction volumes however the alternating nature of the system may introduce a bias in data due to lack of continuous flow of metabolites.

The reliance of existing technologies on passive diffusion of molecules or alternating mixing strategies is not efficient and may not replicate a real-world environment where bi-directional mixing would continuously take place. These methods may also introduce a bias in the study of cell-to-cell interactions.

The preceding discussion of the background to the invention is intended only to facilitate an understanding of the present invention. It should be appreciated that the discussion is not an acknowledgment or admission that any of the material referred to was part of the common general knowledge in the art as at the priority date of the application.

### SUMMARY OF THE INVENTION

In accordance with the invention there is provided a co-culture bioreactor comprising:
at least two chambers, each chamber configured to house a distinct cell type in a shared culture medium and each chamber having an inlet and an outlet for the shared culture medium;
a selectively permeable membrane associated with each of the at least two chambers and in fluid communication with the outlet of one chamber and an inlet of one or more other chambers, each of the selectively permeable membranes being individually configured to prevent permeation of cells from the one chamber it is associated with through the membrane and into the one or more other chambers whilst allowing culture medium and selected biomolecules to permeate therethrough; and
a pump associated with each of the at least two chambers which is configured to circulate culture medium through the selectively permeable membrane and between the at least two chambers.

The at least two chambers may include a first chamber and a second chamber, with a first selectively permeable membrane associated with the first chamber and a second selectively permeable membrane associated with the second chamber, and a first pump in fluid communication with the outlet of the first chamber and the inlet of the second chamber and configured to pump culture medium in the first chamber across the first selectively permeable membrane such that a first permeate stream, which includes culture medium devoid of a first cell type, from the first electively permeable membrane is formed and directed to the inlet of the second chamber in use and a second pump in fluid communication with an outlet of the second chamber and an inlet of the first chamber and configured to pump culture medium in the second chamber across the second selectively-permeable membrane such that a second permeate stream, which includes culture medium devoid of a second cell type, from the second selectively permeable membrane is formed and directed to the inlet of the first chamber in use.

More than two chambers, or reactors, may be provided, each chamber having at least one selectively permeable membrane and at least one pump associated with it, wherein each pump is configured to pump culture medium from the chamber the pump is associated with across the selectively permeable membrane associated with the chamber to cause a culture medium permeate stream substantially devoid of cells to be formed and directed into the one or more other chambers in use. Each pump may be selectively or individually controllable and configured to pump culture medium from the chamber the pump is associated with across the selectively permeable membrane associated with the chamber. At the selectively permeable membrane a permeate stream and a cell-containing culture medium stream may be formed in use. Each cell-containing culture medium stream may be returned to the chamber of the associated membrane, optionally through a further selectively permeable membrane. Each permeate stream may be directed to one or more other chambers, optionally through a further selectively permeable membrane.

Each of the selectively permeable membranes may be configured for crossflow filtration and provide a pressurised interface of a selected size for culture medium and selected biomolecule exchange between chambers in use. Each of the selectively permeable membranes may be housed within a separate membrane enclosure. Each membrane enclosure may include an inlet for culture medium that is in fluid communication with its associated chamber; an outlet for a cell-containing culture medium stream on a feed side of the membrane that is in fluid communication with the associated chamber for returning the cell-containing culture medium stream which includes cells excluded by the selectively permeable membrane to the associated chamber, and a permeate outlet on a permeate side of the membrane for the permeate stream to be directed to one or more other chambers not associated with the membrane in use. The membrane enclosure may be a generally cylindrical housing. The selectively permeable membrane may be generally cylindrical and extend at least partially between the ends of the cylindrical housing with the inlet for the culture medium and outlet for the cell-containing culture medium stream located at opposite ends of the cylindrical membrane. The permeate outlet for the permeate stream may be provided in a side wall of the cylindrical housing for extracting permeate located on the permeate side of the membrane between the membrane and the side wall of the housing in use. The permeate outlet may be near the end of the housing which has the outlet for the cell-containing culture medium stream. The housing may be a stainless-steel housing.

The inlet for the culture medium and the outlet for the cell-containing culture medium stream of the membrane enclosure may be connected through a pump to the associated chamber to permit culture medium to be pumped from the chamber, across the selectively permeable membrane to create a cell-containing culture medium stream that is directed back into the associated chamber and a permeate stream that is directed to one or more other chambers in use. The pump may be configured to continually pump culture medium across the selectively permeable membrane and thus to continually transfer permeate between the two or more different chambers and cell-containing culture medium back into the associated chamber in use. A valve may be provided between the outlet for the cell-containing culture medium stream of the membrane enclosure and the associated chamber, the valve being configured to increase the fluid flow pressure at the selectively permeable membrane to a level at which culture medium and selected biomolecules permeate therethrough.

Each selectively permeable membrane may have pores of an individually selected pore size. The pore size may be selected based on the size of the cell type to be prevented from permeating therethrough and/or the size of the selected biomolecules to be allowed to permeate therethrough. The individually selected pore size may range between about 0.05 μm and 0.2 μm, preferably between about 0.08 μm and 0.1 μm. The membrane may be a ceramic membrane.

A second selectively permeable membrane may be provided downstream of a first selectively permeably membrane associated with a chamber, optionally having a smaller pore size than the first selectively permeable membrane.

Each chamber may have a volume of about 300 ml or more. Each chamber may include a stirrer. Each chamber may include a temperature controller. Each chamber may include a sampling port for sampling culture medium and/or harvesting cells therefrom.

In accordance with a second aspect of the invention, there is provided a co-culture system comprising
a co-culture bioreactor as defined above; and
a controller configured to be in communication with each of the individually controllable pumps and configured to separately or jointly issue machine-readable instructions to each pump to control fluid flow rate of the pump and thus the fluid flow pressure across a selectively permeable membrane associated with the pump.

The controller may further be in communication with a temperature controller associated with each chamber to issue machine-readable instructions to the temperature controller to control the culturing temperature of the associated chamber. The controller may further be in communication with stirrer associated with each chamber to issue machine-readable instructions to the stirrer to control the agitation rate or stirring rate of the associated chamber.

Embodiments of the invention will now be described, by way of example only, with reference to the accompanying drawings.

### BRIEF DESCRIPTION OF THE DRAWINGS

In the drawings:

FIG. 1 is a schematic illustration of a first embodiment of the co-culture bioreactor;

FIG. 2 is a schematic illustration of a second embodiment of the co-culture bioreactor;

FIG. 3 is a schematic illustration of a third embodiment of a co-culture bioreactor;

FIG. 4 is a schematic illustration of a membrane enclosure (left) and a selectively permeable membrane (right) with solid arrows representing cell-containing culture medium as the cells are unable to penetrate through the pores of the selectively permeable membrane and dotted arrows indicate culture media devoid of cells following filtration through the selectively permeable membrane;

FIG. 5 is a schematic illustration of a fourth embodiment of a co-culture bioreactor;

FIG. 6 is a graph showing the change in concentration of glucose and fructose over time in vessels A and B;

FIG. 7 is a graph showing the change in concentration of acetic acid over time in vessels A and B;

FIG. 8 is a graph showing the change in concentration of glycerol over time in vessels A and B;

FIG. 9 is a graph showing the change in concentration of ethanol over time in vessels A and B;

FIG. 10 is a graph showing the change in concentration of total protein over time in vessels A and B;

FIG. 11 is a graph of the change in concentration of amino acids over time in vessels A and B;

FIG. 12 is a graph showing the growth of S. cerevisiae and C. sorokiniana over time in vessels A and B;

FIG. 13 is a graph showing the growth of S. cerevisiae and L. thermotolerans over time in vessels A and B;

FIG. 14 is a graph showing the growth of S. cerevisiae and L. plantarum over time in vessels A and B;

FIG. 15 is a graph showing the OD₆₀₀ and OD₇₅₀ of S. cerevisiae and C. sorokiniana over 36 hours of co-culturing;

FIG. 16 is a graph showing the concentration of glycerol in yeast-containing and algae-containing vessels;

FIG. 17 is a graph showing the concentration of glucose in yeast-containing and algae-containing vessels; and

FIG. 18 is a graph showing the concentration of ethanol in yeast-containing and algae-containing vessels.

### DETAILED DESCRIPTION WITH REFERENCE TO THE DRAWINGS

A multi-membrane bioreactor is provided that allows for the co-culturing of two or more different cell types without any cell-to-cell contact. As used herein the term “different cell types” may refer to cells of two are more different organisms of different microbial species and/or different kingdoms or cells of the same organism that are morphologically or phenotypically distinct or at different life stages.

The co-culture bioreactor includes at least two culture chambers, vessels, or reactors, each configured to house a distinct cell type in a shared culture medium and each having at least an inlet and an outlet for the shared culture medium. A selectively permeable membrane is associated with each of the at least two chambers and in fluid communication with the outlet of one chamber that the membrane is associated with and an inlet of one or more other chambers. Each of the at least two selectively permeable membranes may be individually configured to prevent permeation of cells from the one chamber it is associated with (“the associated chamber”) into the one or more other chambers (“non-associated chambers”) whilst allowing culture medium and selected biomolecules to permeate therethrough. A pump is associated with each of the at least two chambers and is arranged to circulate culture medium across or through the selectively permeable membrane and between the at least two chambers.

The co-culture bioreactor includes parallel pumping circuits for each independently cultured cell type. The exchange of selected biomolecules such as proteins and metabolites between the different culture chambers or reactors of the co-culture bioreactor occurs at each of the selectively permeable membranes. The selectively permeable membranes may be configured for crossflow filtration and may provide a pressurised interface of a selected size for culture medium and biomolecule exchange between chambers. The size of the membrane surface may be increased to enhance the efficiency of culture medium and biomolecule exchange.

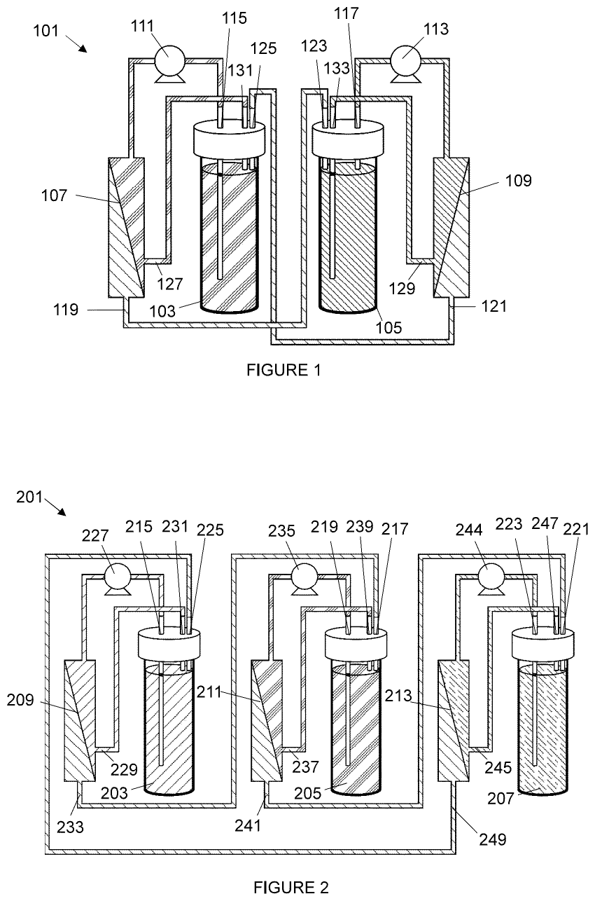

An embodiment of the co-culture bioreactor (101) comprising two culturing chambers is shown in FIG. 1. The co-culture bioreactor includes a first chamber (103) and a second chamber (105), with a first selectively-permeable membrane (107) associated with the first chamber (103) and a second selectively-permeable membrane (109) associated with the second chamber (105). A first pump (111) for pumping culture medium in the first chamber (103) through an outlet (115) for the culture medium in the chamber and across the first selectively-permeable membrane (107) and a second pump (113) for pumping culture medium in the second chamber (105) through an outlet (117) and across the second selectively-permeable membrane (109).

A first permeate (119) from the first membrane (107), which comprises culture medium which should be devoid of a cell type, is directed to the second chamber (105) and enters the second chamber through a shared culture medium inlet (123) provided therein. A second permeate (121) from the second membrane, which should also include culture medium devoid of a cell type, is directed to the first chamber (103) through a shared culture medium inlet (125) provided therein. In this manner, culture medium and selected biomolecules such as metabolites, signalling molecules and proteins is shared between the first chamber (103) and the second chamber (105) without any contact between the different cell types cultured in each of the two chambers.

Each pump (111, 113) may be selectively or individually controllable to pump culture medium from its associated chamber (103, 105) across its associated membrane (107, 109) to cause a permeate (119, 121) stream and a cell-containing culture medium stream (127, 129) to be formed on either side of the membrane (107, 109). The cell-containing culture medium stream (127) is returned to the chamber (103) that the cells are derived from through a cell-containing culture medium inlet (131) provided therein. The return of cell-containing culture medium may occur through a further selectively permeable membrane. Similarly, the cell-containing culture medium stream (129) created at the second selectively permeable membrane (109) is returned to the second chamber (105) that the cells are derived from through a cell-containing culture medium inlet (133) provided therein.

The permeate stream (119) from the first selectively permeable membrane (107) is directed to the other chamber not associated with the particular membrane (107), i.e. the second chamber (105). The permeate stream (121) from the first selectively permeable membrane (109) is directed to the other chamber not associated with the particular membrane (109), i.e. the first chamber (103). The permeate of each of the selectively permeable membranes may optionally be directed to the other chamber not associated with said membrane through a further selectively permeable membrane as will be described in more detail below.

More than two chambers, or reactors, may be provided each having at least one membrane and at least one pump associated with it. An embodiment of the co-culture bioreactor (201) comprising three chambers or reactors is shown in FIG. 2. The co-culture bioreactor (201) includes a first chamber (203), a second chamber (205) and a third chamber (207), each configured to house a distinct cell type in a shared culture medium, with a first selectively permeable membrane (209) associated with the first chamber (203), a second selectively-permeable membrane (211) associated with the second chamber (205) and a third selectively permeable membrane (213) associated with the third chamber (207).

The first selectively permeable membrane (209) is in fluid communication with a culture medium outlet (215) of the first chamber (203) and a shared culture medium or permeate inlet (217) of the second chamber (205). The second selectively permeable membrane (211) is in fluid communication with a culture medium outlet (219) of the second chamber (205) and a permeate inlet (221) of the third chamber (207). The third selectively permeable membrane (213) is in fluid communication with a culture medium outlet (223) of the third chamber (207) and a permeate inlet (225) of the first chamber (203). Each of the selectively permeable membranes (209, 211, 213) are individually configured to prevent permeation of cells of the distinct cell type from its associated chamber into the one or more other chambers whilst allowing culture medium and selected biomolecules to permeate therethrough.

A first individually controllable pump (227) is provided for pumping or circulating culture medium in the first chamber (203) through the outlet (215) and across the first selectively-permeable membrane (209) to create a first cell-containing culture medium stream (229) which is redirected back to the first chamber (203) through the cell-containing culture medium stream inlet (231) and a permeate stream (233) which is directed to the second chamber (207) through the permeate inlet (217). A second individually controllable pump (235) is provided for pumping or circulating culture medium in the second chamber (205) through the outlet (219) and across the second selectively-permeable membrane (211) to create a second cell-containing culture medium stream (237) which is redirected back to the second chamber (205) through the cell-containing culture medium stream inlet (239) and a permeate stream (241) which is directed to the third chamber (207) through the permeate inlet (221) of the third chamber. A third individually controllable pump (244) is provided for pumping or circulating culture medium in the third chamber (207) through the outlet (223) and across the third selectively-permeable membrane (213) to create a third cell-containing culture medium stream (245) which is redirected back to the third chamber (207) through the cell-containing culture medium stream inlet (247) and a permeate stream (249) which is directed to the first chamber (203) through the permeate inlet (225) of the first chamber (203).

In this embodiment the culture sharing between the three chambers (201, 203, 205) occurs in series with the permeate from each selectively permeable membrane being transferred to a single other chamber, not associated with the membrane. However, the permeate may also be directed to both other chambers not associated with the membrane, i.e., in parallel, to increase the efficiency of medium sharing and metabolic exchange between the three different cell cultures.

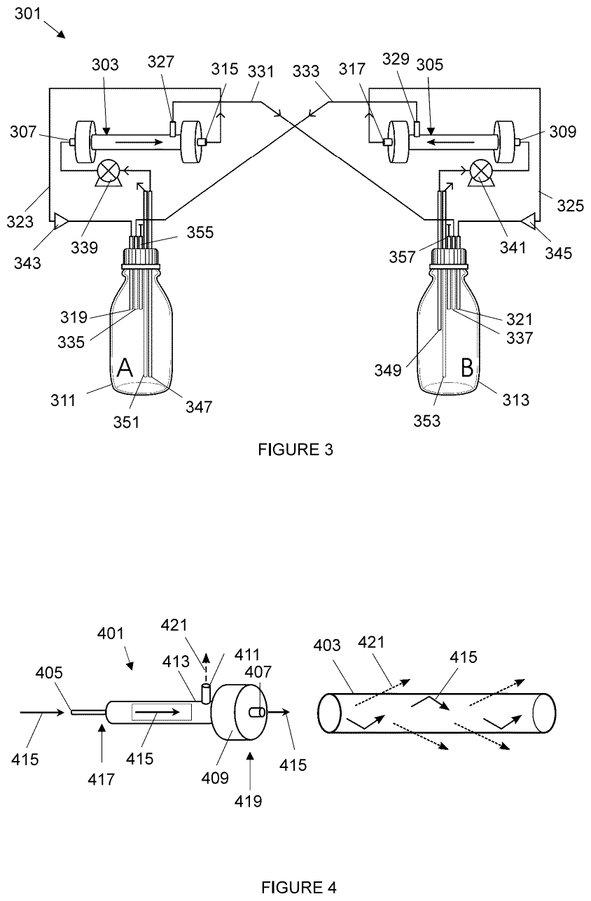

A selectively permeable membrane of the multi-membrane co-culture bioreactor may be housed within a separate membrane enclosure. The membrane enclosures (303, 305) of the two selectively permeable membranes (not shown in FIG. 3) of the embodiment illustrated in FIG. 3, for example, each include an inlet (307, 309) for culture medium that is in fluid communication with the chamber (311, 313) associated with the membrane. Each membrane enclosure (303, 305) also has an outlet (315, 317) for the cell-containing culture medium stream on a feed side of the membrane that is in fluid communication with the cell containing culture medium inlet (319, 321) of the associated chamber (311, 313) for returning the cell-containing culture medium stream (323, 325) to the associated chamber (311, 313). Each membrane enclosure further has a permeate outlet (327, 329) on the permeate side of the membrane for the permeate stream (331, 333) to be directed to the other chamber not associated with the membrane. The respective permeate outlets (327, 329) of the membrane enclosures (303, 305), are each in fluid communication with a permeate or shared culture medium inlet (335, 337) of a non-associated chamber (311, 313).

The membrane enclosure may be a generally cylindrical housing (401) as shown in FIG. 4. The selectively permeable membrane (403) may be generally cylindrical and extend at least partially, but preferably completely, between the ends of the cylindrical housing when assembled within the housing (401). When it extends partially between the ends of the housing, seals or plugs must be provided between the selectively permeable membrane (403) and housing (401) to ensure that cell-containing medium does not bypass the membrane into the permeate stream. The size of the membrane surface of a cylindrical membrane may be increased by elongating the cylindrical membrane, thereby enhancing the efficiency of culture medium and biomolecule exchange. The inlet (405) for the culture medium and outlet (407) for the cell-containing culture medium stream (415) are located at opposite ends of the membrane which may be at or near opposite ends (417, 419) of the cylindrical housing (401). The housing may be provided with one or more endcaps (409) that include the inlet and the outlet (407) for the feed culture medium stream (i.e., the cell-containing stream) on opposite ends of the cylindrical housing (401). The permeate outlet (411) for the permeate stream is provided in a side wall (413) of the cylindrical housing (401) for extracting permeate (421) located on the permeate side of the membrane (403), i.e., the space defined between the membrane (403) and the side wall (413) of the housing (401). The permeate outlet (411) may be near the end (419) of the housing (401) with the outlet for the cell-containing culture medium stream (415). The housing may be made of stainless-steel.

Referring back to FIG. 3, an embodiment of co-culture bioreactor (301) is illustrated which is suitable for co-culturing cells of a first organism A, and a cells of a second organism B, wherein the cells of both organisms are of a similar size. The embodiment may be suitable for co-culturing two different yeast species or yeast and microalgae, for example. The first chamber (311) houses a cell culture of organism A and the second chamber (313) houses a cell culture of organism B. The two modular and thus separately controllable culture chambers or reactors (311, 313) each have an associated selectively permeable membrane housed in a membrane enclosure (303, 305). In this embodiment, the selectively permeable membranes used to physically separate or isolate the cells within their respective culturing chambers (311, 313) have a similar configuration (i.e., similar surface area and pore size) due to the similar cell size of the cell types A and B. In this embodiment, the selectively permeable membrane has a pore size of 0.1 μm.

Each of the inlets (307, 309) for the culture medium and the outlets (315, 317) for the cell-containing culture medium stream of the membrane enclosures (303, 305) are connected through a pump (339, 341) to the associated chamber (311, 313) to permit culture medium to be pumped from the outlet (347, 349) of the chamber (311, 317), across the selectively permeable membrane to create a cell-containing culture medium stream (323) that is directed back into the associated chamber (311, 317) and a permeate stream (331, 333) that is directed through suitable tubing or piping to one or more other chambers. In this embodiment, the individually controllable pumps (339, 341) are set to pump at more or less the same pressure or flow rate due to the similarly configured membranes that are employed to prevent contact between cell types A and B that are of a similar size. Each of the pumps (339, 341) are configured to continually pump culture medium across the selectively permeable membrane and to accordingly, continually transfer permeate (331, 333) (i.e., shared culture medium, optionally including selected biomolecules) between the two different chambers and continually transfer cell-containing culture medium back into the associated chamber.

Valves (343, 345), which may be stainless-steel needle valves, diaphragm valves or the like, are provided in between each of the outlets (315, 317) for the cell-containing culture medium stream and the cell-containing culture medium inlets (319, 321) of the respective chambers (311, 313). The valves (343, 345) are configured to increase the fluid flow pressure at each of the selectively permeable membranes to a level at which culture medium and selected biomolecules permeate therethrough (i.e. a permeate stream forms) whilst cells are excluded by the membrane due to the selected pore size.

The cells excluded by each membrane are washed through the respective membrane enclosures (303, 305) by continuously pumping culture media and returning the cell-containing culture medium stream to the chamber associated with the membrane, thereby preventing build-up of cells on the membranes.

Sampling ports (351, 353) are provided into each chamber (311, 313) for the sterile sampling of culture medium from each of the chambers (311, 313) during a co-culturing process. Each chamber (311, 313) includes a headspace sharing port or a gas outflow port (355, 357) with a filter.

In order to prevent the overflow of the chambers the shared medium outlet (349) or removal tube in one of the chambers (313) extends into the chamber to a selected height/depth in the chamber so that the medium will not be removed from the chamber (313) after a certain volume.

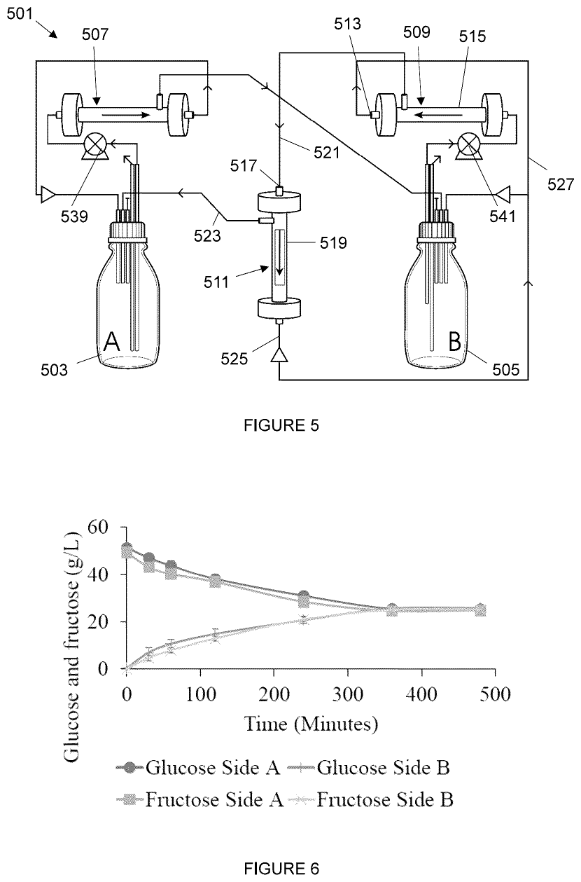

An embodiment of a co-culture bioreactor that is suitable for the co-culture of two different organisms of different cell size is shown in FIG. 5. Organisms of two different kingdoms, such as yeast and bacteria, may be co-cultured with this embodiment, for example. As with previous embodiments, the co-culture bioreactor (501) includes parallel pumping circuits for each independently cultured cell type and has a first chamber (503) for housing organism A (yeast for example) and a second chamber (505) for housing organism B (bacteria for example) with a much smaller cell size than that of organism A. Pumps (539, 541) provided between the chambers (503, 505) and in fluid communication with each of the chambers (503, 505) are arranged to circulate shared culture medium between the chambers, whilst the selectively permeable membranes (507, 509) associated with each chamber (503, 505) avoid sharing of cells between the chambers. The difference between the embodiment shown in FIG. 5 and the embodiment shown in FIG. 3 is that two selectively permeable membranes (509, 511) are associated with the second chamber (505) housing organism B. In this embodiment, the membrane (507) associated with the first chamber (503) and the first membrane (509) associated with the second chamber (505) have the same pore size of about 0.1 μm. The second membrane (511) associated with the second chamber (505) has a pore size of about 0.08 μm. The second membrane (511) is provided downstream of the first membrane (509). The permeate outlet (513) of the first membrane enclosure (515) is in fluid communication with an inlet (517) of the membrane enclosure (519) of the second membrane (511). In this manner a first permeate stream (521) formed at the first membrane (509) when the pump (541) is in operation is directed to the second membrane (511) where a second permeate stream (523) is created that is directed to the first chamber (503), not associated with the membrane, and a second cell-containing culture medium stream (525) is created that gets directed back to the associated second chamber (505). The second cell-containing culture medium stream (525) may be merged with a first cell-containing culture medium stream (527) to be returned to the second chamber (505) together.

When co-culturing yeast and bacteria, for example, the culture medium may be directed across a membrane with a larger pore size first and then a second membrane with a smaller pore size to make sure the pressure drop across the membrane with a smaller pore size is not too high. The pressure on the membrane with smaller pores is reduced by first running the culture medium through the membrane with a larger pore size. The permeate from the first membrane still includes at least some bacterial cells, which are only separated from the shared culture medium at the second membrane with the smaller pore size. A single membrane with an appropriate pore size may be used to obtain cell separation and other suitable pressure reducing means be included in the fluid flow path to prevent a pressure drop across the membrane, should it be necessary.

The selectively permeable membrane of the co-culture bioreactor may be cylindrical in shape and allow for the flow of cell-containing culture media through the cylindrical membrane in a manner that prevents cellular build-up on the internal surface of the membrane. The pressure in the co-culture bioreactor, as the culture medium is pumped or circulated therethrough, forces culture medium devoid of cells to permeate or filtrate through the elongate and cylindrical membrane and exit the permeate port or permeate outlet of the membrane enclosure. The shape and size of the membrane may be optimised to produce an optimal surface area for such crossflow culture medium filtration depending on the type of cells cultured. The selectively permeable membrane may be a ceramic membrane.

The selectively permeable membrane may have pores of a selected pore size. The pore size may be selected based on the size of the cell type to be prevented from permeating therethrough and/or the size of the selected biomolecules to be allowed to permeate therethrough. The pore size may range between about 0.05 μm and 0.2 μm, preferably between about 0.08 μm and 0.1 μm.

Each culture chamber of the co-culture bioreactor may have a volume of about 300 ml or more. Each culture chamber may include a suitable stirring means such as a magnetic or other type of stirrer capable of continuously mixing the contents of the chamber during the culturing process. To avoid overflow of the chambers, a removal tube in at least one of the chambers is placed at a selected volume. Each culture chamber may include a temperature controller. Each culture chamber may include a sampling port for sampling culture medium and/or harvesting cells therefrom.

The cell-containing culture medium and cell-devoid permeate may be circulated through the co-culture bioreactor in tubing or piping with a diameter selected for the desired flow rate to be maintained with the pumps. For example, liquid culture medium may be circulated through the co-culture bioreactor using tubing with an internal diameter of 8 mm providing a flow rate of 4 ml/s using two peristaltic pumps. The tubing may be thermoplastic elastomer tubing, preferably with chemical resistance to oxidising agents, acids and alkalis and resistance to flex-fatigue such as Tygon®, Norprene® tubing (Saint-Gobain Abrasives, Inc).

The co-culture bioreactor may also form part of a co-culture system that further includes a controller configured to be in communication with each of the individually controllable pumps. The controller may separately or jointly issue machine-readable instructions to each pump to control their respective pumping force and thus the fluid flow rate and/or fluid flow pressure across the respective selectively permeable membranes of the co-culture bioreactor. The controller may further be in communication with a temperature controller (with a heating element and thermometer) that is associated with each chamber to issue machine-readable instructions to the temperature controller to control the culturing temperature of the associated chamber. The controller may also be configured to be in communication with agitation means such as a stirrer of each chamber to control the mixing rate in the chamber.

Experiments were carried out to validate the co-culture bioreactor under different conditions and with various microbial species combinations, including co-cultures combining species from vastly different evolutionary backgrounds. In order for effective co-culturing to take place, it must be ensured that a variety of metabolites can pass freely and quickly through the membranes while still preventing the transfer of whole cells between the chambers, without damaging the cells.

### Materials and Methods

### Membrane Permeability Testing

A variety of metabolites were monitored in the multi-membrane system to ensure that effective mixing of co-culture medium took place. A bioreactor of the configuration shown in FIG. 3 was assembled and initially run with distilled water in one vessel and a mixture of metabolites of known concentration in distilled water in the other (Table 1). This test media was used only to determine the movement of specific substances through the membrane. Amino acids were added to the medium at concentrations described by Bely et al. (1990). Three representative amino acids at varying initial concentrations in the medium were chosen as a representative dataset. Both ceramic membranes were of size 0.1 μm and the fully assembled co-culture bioreactor was autoclaved before use. The co-culture bioreactor was run at 50 rpm for eight hours and 20 ml samples from both vessels were taken at various timepoints during the run. These runs were performed in triplicate alternating the distilled water in Vessel A (the first chamber) and Vessel B (the second chamber).

TABLE 1
Metabolites used to test permeability of ceramic membrane.

| | Concentration |
|---|---|
| Metabolite | (g/L) |
| Glucose | 50 |
| Fructose | 50 |
| Acetic acid | 8 |
| Glycerol | 20 |
| Ethanol | 10 |
| Total protein (BSA | 20 |
| and lactalbumin) | |
| Amino acids | |
| Proline | 0.468 |
| Lysine | 0.013 |
| Alanine | 0.111 |

### Nutrient Analysis

Metabolite concentrations were analysed by centrifugation of 2 ml samples at 10 000 g for 5 minutes. The supernatant was removed and stored at −20° C. until the assay was performed according to the protocols provided. Glucose and fructose were measured using the D-Fructose/D-Glucose Assay Kit from Megazyme (Bray, Ireland). Acetic acid was measured using the Acetic Acid Assay Kit (ACS Manual Format) from Megazyme (Bray, Ireland). Glycerol was measured using the Glycerol Assay Kit from Sigma-Aldrich (Missouri, USA). Ethanol was measured using the Ethanol Assay Kit from Sigma-Aldrich (Missouri, USA). Total protein content was measured using the Bradford Reagent assay protocol supplied by Sigma-Aldrich (Missouri, USA).

### Amino Acid Analysis

All reagents, standards and solvents were purchased from Sigma-Aldrich (St. Louis, MO, USA) and used with-out further purification. Water was obtained from a Milli-Q filtration system (Millipore Filter Cor., Bedford, MA, USA) and degassed before used. The amino acid analyses were performed with a 1260 infinity Agilent HPLC (Agilent, Palo Alto, CA, USA) equipped with a 1260 DAD and FLD detector. Both the online derivatization and instrumental method used were based on the Agilent 5991-5571 application note with some modifications. The modifications include calibration range (0.1-50 mg/L) using Norvaline as the internal standard (20 mg/L), LOQ of 0.05 mg/L LOD of 0.015 mg/L.

### Separation Testing: Pre-Culturing and Co-Culturing of Microorganisms in the Multi-Membrane System

Three different co-cultures were tested using the multi-membrane system, namely yeast-yeast, yeast-microalgae, and yeast-bacteria. Lachancea thermotolerans, Chlorella sorokiniana and Lactobacillus plantarum were paired with Saccharomyces cerevisiae. Precultures of each species were grown as monocultures in their respective media (Table 2) in 50 ml volumes until cultures reached mid-log phase. Cell concentrations were established using optical density measurements prior to inoculation. Precultures were inoculated at an OD₆₀₀ and OD₇₅₀ of 0,1 for yeast and microalgae respectively, into 1,8 L of autoclaved media and then these vessels were placed within the autoclaved multi membrane system. Each combination of microbial species was performed in triplicate.

TABLE 2
Preculture and co-culture media for multi-membrane system experiments

| | | Optical density | Co-culture media |
|---|---|---|---|
| Species | Preculture media | measurement | (with S. cerevisiae) |
| Saccharomyces cerevisiae | Yeast extract- | OD₆₀₀ | N/A |
| | peptone-dextrose | | |
| | (YPD) medium | | |
| Chlorella sorokiniana | Tris-acetate | OD₇₅₀ | Tris-acetate |
| | phosphate (TAP) | | phosphate (TAP) |
| | medium (Gorman and | | medium (Gorman and |
| | Levine 1965) | | Levine 1965) |
| | containing 1x Hom's | | containing 1x Hom's |
| | vitamins (personal | | vitamins (personal |
| | communication, Erik | | communication, Erik |
| | F. Y. Hom 2015) | | F. Y. Hom 2015) with |
| | | | added 2% glucose |
| Lactobacillus plantarum | De Man, Rogosa and | OD₆₀₀ | Yeast extract- |
| | Sharpe (MRS) agar | | peptone-dextrose |
| | | | (YPD) medium |
| Lachancea thermotolerans | Yeast extract- | OD₆₀₀ | Yeast extract- |
| | peptone-dextrose | | peptone-dextrose |
| | (YPD) medium | | (YPD) medium |

Growth of microbial species was measured using haemocytometer cell counts every 12 hours for 36 hours for yeast and microalgal co-culturing and plate counts for yeast and bacterial co-culturing. Samples were also plated out from vessels onto WL, MRS and TAP media to ensure no movement of species between vessels or contamination had taken place. These plates were incubated at 25° C. for 3 days before counting.

Yeast-Microalgae System with Indirect Contact

The yeast-microalgae bioreactor illustrated in FIG. 3 was selected for further metabolic analysis to observe the production and utilization of various metabolites in an indirect contact experiment. Growth of both species were monitored using OD₆₀₀ and OD₇₅₀ for S. cerevisiae and C. sorokiniana, respectively. Samples were plated out every hour for the first 12 hours and every 6 hours thereafter to check for any movement of partner species across the membrane. Glucose concentrations were measured using the photometric determination of glucose (mg/L) in sample material based on the Enzytec fluid glucose method and performed on a Thermo Scientific Arena 20XT Analyzer. Glycerol concentrations were measured using the photometric determination of glucose (mg/L) in sample material based on the Enzytec fluid glycerol method and performed on a Thermo Scientific Arena 20XT Analyzer.

### Results

### Membrane Permeability Analysis of Various Metabolites

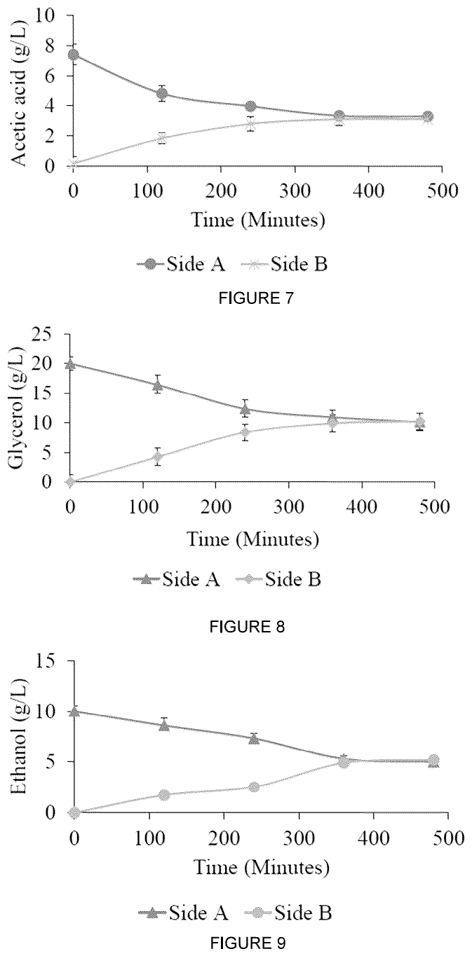

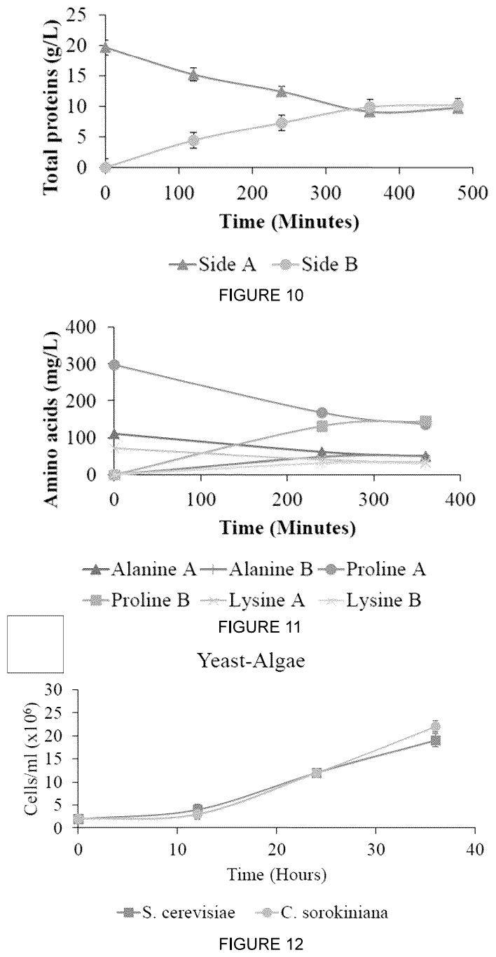

For effective co-culturing to take place it must be ensured that a variety of metabolites can pass freely through the membranes while still preventing the transfer of whole cells between the vessels. This allows media free of cells to be passed between the vessels and simulates an environment wherein cells can exchange metabolic products without direct physical contact. To test this vessel A was prepared to contain a variety of metabolites to simulate those present in media typic for co-culture of yeast, bacteria and microalgae and produced by cell culture such as sugars, proteins, amino acids, and alcohols while Vessel B contained only distilled water. The system was set up to run over 8 hours and 10 ml volumes were sampled regularly at intervals of 10 minutes for the first 2 hours and every 30 minutes thereafter. These samples were stored at −20° C. until metabolic assays could be completed. These samples were tested using commercial enzyme assay kits to monitor changes in concentrations of metabolites over time. The movement of metabolites was analysed to ensure that equal mixing of the vessels could take place during co-culturing and to determine the point of equalization in both vessels. The metabolites measured display a clear pattern of mixing between the two vessels and equalization was shown to take place after 6 hours of co-culturing as shown in FIGS. 6 to 10. Amino acids also showed equalization after 6 hours of co-culturing at all concentrations shown in FIG. 11.

### Separation Testing of Multi-Membrane System

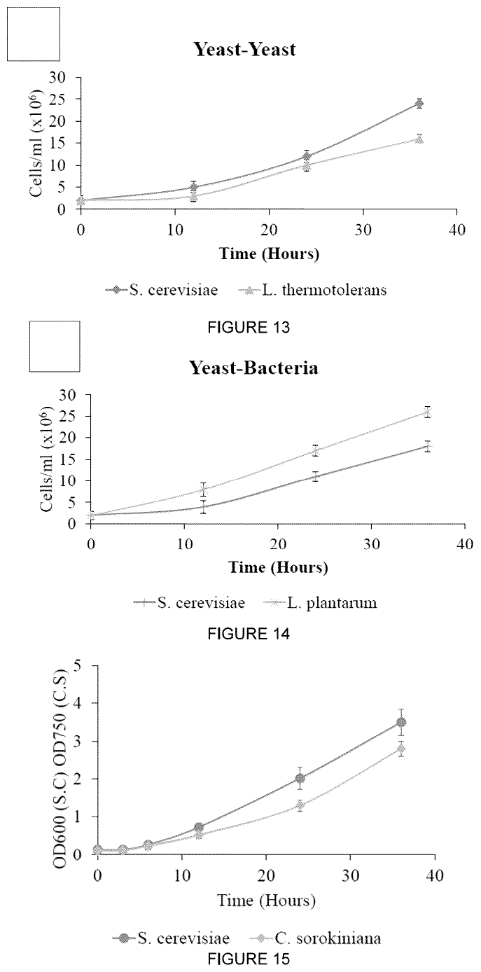

Growth of microalgal, yeast and bacterial species was monitored using either haemocytometer counting techniques (yeast-microalgae system) or using plate counts (yeast-bacterial and yeast-yeast systems). The plates were incubated at 25° C. for 3 days before counting took place. Plating out of cultures also served as a contamination check for the system and culture samples were checked microscopically. Samples from vessels were also plated out onto agar supporting the growth of the microorganisms in the partner vessel to ensure no mixing of cultures took place while the system was running. All species grown in indirect contact were able to proliferate after inoculation into separate vessels (FIGS. 12 to 14). The presence of the membrane and pressure applied to the system did not prevent cell growth.

Yeast-Microalgae System with Indirect Contact Between Species

The yeast-microalgae indirect contact system underwent further characterisation to assess the efficient exchange of metabolites in co-culture conditions. Glucose, glycerol, ethanol, and acetic acid were monitored during co-culture. Samples were plated out throughout co-culturing to ensure no movement of species had occurred across the membrane.

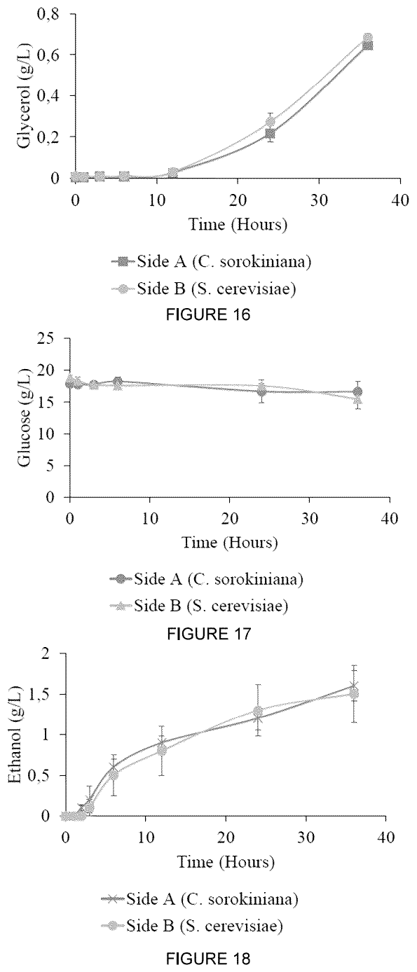

S. cerevisiae and C. sorokiniana grew to an OD of between 2,7 and 3,5 over 36 hours of co-culturing (FIG. 15). The growth rate of C. sorokiniana is consistent with that found in previous studies using this winery wastewater isolate. Glycerol (FIG. 16), produced by S. cerevisiae, increased over time, and the metabolite was efficiently exchanged between the two compartments since concentrations on both sides remained similar throughout the duration of the experiment, with only a small difference in the timing of the accumulation in line with the time required for the transfer from the producing compartment to the receiving compartment. Ethanol followed a similar pattern to glycerol, but a slightly larger difference of 10-15% in concentration between the two compartments was observed. This may be due to continuous use of ethanol by C. sorokiniana, resulting in a continuous disequilibrium and a flow from the yeast to the algal bioreactor. This also highlights that no such system can ever be in a perfect balance, in particular when producer and user organisms are co-cultured. Glucose was found at a concentration of 19 g/L in both vessels and was utilized to a final concentrate of approximately 15 g/L in both vessels after 36 hours of co-culturing (FIGS. 17 and 18). Glucose can be used a carbon source by both species however the concentration remained consistent between the two vessels implying that sufficient mixing of media was taking place.

The results demonstrate that the co-culture bioreactor efficiently and actively mixes media between two chambers using membrane filters and that metabolic equalization occurs after only six hours using an experimental setup. Species are kept separate while sharing their produced metabolites and growth media, which is useful for co-culture and consortia studies. Metabolites of various sizes and concentrations were tested such as amino acids, sugars and ethanol. The system could be run for a period of up to 36 hours and the pressure applied to force media through the membrane did not prevent the proliferation of either species. A variety of species were used for co-culturing indicating the wide variety of applications the bioreactor could have. The results further demonstrate that the co-culture bioreactor can effectively be used in inter-species and inter-kingdom microbial studies. The bioreactor may provide the ability to clarify the effects of metabolic contact in relation to cell contact between species.

The co-culture bioreactor addresses many of the drawbacks of existing systems since it allows continuous and efficient exchange of metabolites and proteins without disruption of flow, and by not relying on passive diffusion alone. Since culture medium transfer between the chambers is relatively fast, concentration gradients do not build up. The multi-membrane co-culture bioreactor utilizes active flow of media from both chambers into the other and complete mixing occurs after only 6 hours of co-culture in the exemplary bioreactor tested. Bioreactors running at higher pressures or with larger membrane surface areas may result in a quicker equalization of media. The co-culture bioreactor allows for rapid exchange of metabolites and proteins between two or more chambers, ensuring shared medium composition between the chambers while completely separating the organisms or cell types within these chambers. The co-culture bioreactor may be used to improve the understanding of the effects of physical cell contact between microbial species and allow for their efficient exploitation in both research and industry. At laboratory scale, it may allow experimenters to study the interaction of metabolism separate from physical interactions.

The co-culture bioreactor is modular allowing for separate and different fermentation conditions for co-culture. Since the reactor volumes of the fermentation chambers are separate, control variables like temperature or the like can be independently controlled in respect of each chamber. This means that different species may be co-cultured with the bioreactor which may have not been co-cultured before.

The modularity also allows for the customization or variation of chamber size and the fluid flow and pressure in the system. The volumes of the chambers can easily be scaled and the transfer rate of the media controlled as a function of the membrane area. The membrane surface area is also easily scalable. The co-culture bioreactor also allows for larger liquid volumes to be sampled than existing transwell systems for downstream analyses. The co-culture bioreactor can be scaled to various sizes and can be extended to include more than two independent culture chambers.

The co-culture bioreactor is particularly useful for co-fermentation of yeast species. The transfer between chambers is of a speed sufficient that biomolecules such as the metabolites of one species are transferred to the other chamber for next-step fermentation. The co-culture bioreactor allows for continuous one-directional flow without disruption and without need for regular backwashes. In particular, the use of cylindrical membranes in membrane enclosures allows flowthrough of media to prevent the build-up of microbial cells on the membrane surface.

The foregoing description has been presented for the purpose of illustration; it is not intended to be exhaustive or to limit the invention to the precise forms disclosed. Persons skilled in the relevant art can appreciate that many modifications and variations are possible in light of the above disclosure.

The language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

Finally, throughout the specification and accompanying claims, unless the context requires otherwise, the word ‘comprise’ or variations such as ‘comprises’ or ‘comprising’ will be understood to imply the inclusion of a stated integer or group of integers but not the exclusion of any other integer or group of integers.

1. A co-culture bioreactor comprising
at least two chambers, each chamber configured to house a distinct cell type in a shared culture medium and each chamber having an inlet and an outlet for the shared culture medium;
a selectively permeable membrane associated with each chamber and in fluid communication with the outlet of one chamber and the inlet of one or more other chambers, each of the selectively permeable membranes being individually configured to prevent permeation of cells from the one chamber it is associated with through the membrane and into the one or more other chambers whilst allowing culture medium and selected biomolecules to permeate therethrough; and
a pump associated with each chamber which is configured to circulate culture medium through the selectively permeable membrane and between the at least two chambers.

2. The co-culture bioreactor as claimed in claim 1, including a first chamber and a second chamber, with a first selectively permeable membrane associated with the first chamber and a second selectively permeable membrane associated with the second chamber, and a first pump in fluid communication with the outlet of the first chamber and the inlet of the second chamber and configured to pump culture medium in the first chamber across the first selectively permeable membrane such that a first permeate stream from the first selectively permeable membrane is formed and directed to the inlet of the second chamber and a second pump in fluid communication with an outlet of the second chamber and an inlet of the first chamber and configured to pump culture medium in the second chamber across the second selectively-permeable membrane such that a second permeate stream from the second selectively-permeable membrane is formed and directed to the inlet of the first chamber in use.

3. The co-culture bioreactor as claimed in claim 1, including more than two chambers, each chamber having at least one selectively permeable membrane and at least one pump associated with it, and wherein each pump is configured to pump culture medium from the chamber the pump is associated with across the selectively permeable membrane associated with the chamber to cause a culture medium permeate stream substantially devoid of cells to be formed and directed into the one or more other chambers in use.

4. The co-culture bioreactor as claimed in claim 1, wherein each pump is individually controllable and configured to pump culture medium from the chamber the pump is associated with across the selectively permeable membrane associated with the chamber.

5. The co-culture bioreactor as claimed in claim 1, wherein each of the selectively permeable membranes are configured for crossflow filtration and provide a pressurised interface of a selected size for culture medium and selected biomolecule exchange between chambers in use.

6. The co-culture bioreactor as claimed in claim 1, wherein each of the selectively permeable membranes is housed in a membrane enclosure with an inlet for culture medium that is in fluid communication with its associated chamber; an outlet for a cell-containing culture medium stream on a feed side of the membrane that is in fluid communication with the associated chamber for returning a cell-containing culture medium stream to the associated chamber in use, and a permeate outlet on the permeate side of the membrane for the permeate stream to be directed to one or more other chambers not associated with the membrane in use.

7. The co-culture bioreactor as claimed in claim 6, wherein the membrane enclosure is a generally cylindrical housing and the selectively permeable membrane is generally cylindrical and extends at least partially between the ends of the cylindrical housing with the inlet for the culture medium and outlet for the cell-containing culture medium stream located at opposite ends of the cylindrical membrane.

8. The co-culture bioreactor as claimed in claim 6, wherein the permeate outlet is provided in a side wall of the cylindrical housing for extracting permeate located on the permeate side of the membrane between the membrane and the side wall of the housing in use.

9. The co-culture bioreactor as claimed in claim 8, wherein the permeate outlet is near the end of the cylindrical housing which has the outlet for the cell-containing culture medium stream.

10. The co-culture bioreactor as claimed in claim 6, wherein the inlet for the culture medium and the outlet for the cell-containing culture medium stream of the membrane enclosure are connected through the pump to the associated chamber to permit culture medium to be pumped from the chamber, across the selectively permeable membrane to create the cell-containing culture medium stream which is directed back into the associated chamber and the permeate stream which is directed to one or more other chambers, and wherein the pump is configured to continually pump culture medium across the selectively permeable membrane and thus to continually transfer permeate between the two or more different chambers and return cell-containing culture medium into the associated chamber.

11. The co-culture bioreactor as claimed in claim 6, which includes a valve between the outlet for the cell-containing culture medium stream of the membrane enclosure and the associated chamber, the valve being configured to increase the fluid flow pressure at the selectively permeable membrane to a level at which culture medium and selected biomolecules permeate therethrough.

12. The co-culture bioreactor as claimed in claim 1, wherein each selectively permeable membrane has an individually selected pore size ranging between about 0.05 μm and 0.2 μm.

13. The co-culture bioreactor as claimed in claim 12, wherein each selectively permeable membrane has an individually selected pore size ranging between about 0.08 μm and 0.1 μm.

14. The co-culture bioreactor as claimed in claim 1, wherein the selectively permeable membrane is a ceramic membrane.

15. The co-culture bioreactor as claimed in claim 1, wherein a second selectively permeable membrane is provided downstream of a first selectively permeably membrane associated with a chamber, optionally having a smaller pore size than the first selectively permeable membrane.

16. The co-culture bioreactor as claimed in claim 1, wherein each chamber has a volume of about 300 ml or more.

17. The co-culture bioreactor as claimed in claim 1, wherein each chamber has an individually controllable stirrer.

18. The co-culture bioreactor as claimed in claim 1, wherein each chamber has an individually controllable temperature controller.

19. The co-culture bioreactor as claimed in claim 1, wherein each chamber includes a sampling port for sampling culture medium or harvesting cells therefrom.

20. A co-culture system comprising
a co-culture bioreactor as claimed in claim 1; and
a controller configured to be in communication with each individually controllable pump and configured to separately or jointly issue machine-readable instructions to each pump to control fluid flow rate of the pump and thus the fluid flow pressure across a selectively permeable membrane associated with the pump.

21. The co-culture system as claimed in claim 20, wherein the controller is in communication with a temperature controller associated with each chamber to issue machine-readable instructions to the temperature controller to control the culturing temperature of the associated chamber, and wherein the controller is in communication with a stirrer associated with each chamber to issue machine-readable instructions to the stirrer to control the stirring rate of the associated chamber.
